(19) United States
(12) Patent Application Publication
(10) Pub. No.: US 2020/0190567 A1
(43) Pub. Date: Jun. 18, 2020

(54) Method For Detecting Activity Change Of Transposon In Plant Before And After Stress Treatment

(71) Applicant: Beijing Forestry University, Beijing (CN)

(72) Inventors: Deqiang Zhang, Beijing (CN); Yiyang Zhao, Beijing (CN); Jianbo Xie, Beijing (CN)

(73) Assignee: Beijing Forestry University, Beijing (CN)

(21) Appl. No.: 16/564,135

(22) Filed: Sep. 9, 2019

(30) Foreign Application Priority Data
Dec. 17, 2018 (CN) 201811542646.7

(51) Int. Cl. C12Q 1/6869; C12Q 1/6895; G16B 30/00; G16B 40/10; G16B 20/20

(52) U.S. Cl. CPC C12Q 1/6869; C12Q 1/6895; G16B 30/00; C12Q 2600/13; G16B 20/20; C12Q 2600/178; C12Q 2600/158; G16B 40/10

(57) ABSTRACT

The present invention relates to the technical field of genetics and provides a method for detecting activity change of a transposon in a plant before and after stress treatment. The method includes the following steps: 1) respectively extracting total RNAs of samples before and after stress treatment; 2) respectively constructing cDNA libraries of the samples before and after stress treatment by using the total RNAs; 3) sequencing the cDNA libraries; 4) respectively screening siRNAs from raw sequencing data, and combining the screened siRNAs to obtain a total siRNA, and performing cluster clustering on the total siRNA; 5) extracting repeat in whole genome data by using repeatmasker software to obtain positional information of the plant whole genome transposon; and 6) obtaining activity change in the transposon of the plant before and after treatment by means of change in siRNA cluster expression quantity. The method fills the technical gap in the field of plant transposon activity detections.

This application claims priority to Chinese application number 201811542646.7, filed Dec. 17, 2018, with a title of METHOD FOR DETECTING ACTIVITY CHANGE OF TRANSPOSON IN PLANT BEFORE AND AFTER STRESS TREATMENT. The above-mentioned patent application is incorporated herein by reference in its entirety.

### TECHNICAL FIELD

The present invention relates to the technical field of genetics, and in particular, to a method for detecting activity change of a transposon in a plant before and after stress treatment.

### BACKGROUND

DNA sequencing technology is the most important experimental technology in genomics and has a wide range of applications in the entire field of biology. An end-termination sequencing method invented by Sanger in 1977 is a milestone for genome sequencing research. The Sanger method is simple and rapid, and has been improved to become the main method of DNA sequencing research. With the development of genomics science, the traditional Sanger sequencing method can no longer meet the needs of scientific research. To meet these research needs, the second-generation high-throughput sequencing technology is emerged at the right moment and developed rapidly. The genetic principle of the second-generation high-throughput sequencing technology is sequencing by synthesis, i.e., by capturing newly synthesized end-labels to determine DNA sequences. Based on the Sanger sequencing method, four dNTPs are labeled with different colors of fluorescents. When the complementary strand is synthesized by DNA polymerase, different fluorescents are released when each dNTP is added, which is processed by a specific computer software according to the captured fluorescent signal, thereby obtaining sequence information of a DNA to be tested.

A transposon, also known as a jumping factor, is essentially a DNA fragment of a certain length because it can “jump” from one locus of the chromosome to another locus in the genome of an organism, or from one chromosome to another chromosome. The discovery of plant transposons has profound significance for the development of molecular biology. The application of the high-throughput sequencing technology in the transposon research mainly focuses on estimating the content of transposons, target site preference and distribution of transposons, polymorphism of transposons and population frequency, horizontal transfer of transposons and other researches. Although the transposon plays a significant role in various aspects such as plant growth and development, physiological responses, and gene expression, it is difficult to calculate the activity change in the transposon due to the moving characteristics of the transposon. Therefore, it is difficult to directly analyze the transposon activity with the sequencing technology.

### SUMMARY

In view of the above, an objective of the present invention is to provide a method for detecting activity change of a transposon in a plant before and after stress treatment, which solves the problem that the activity change of a transposon in a plant cannot be identified in the prior art.

To achieve the above purpose, the present invention provides the following technical solution.

A method for detecting activity change of a transposon in a plant before and after stress treatment includes the following steps:

1) extracting total RNAs of a sample before stress treatment and after stress treatment, respectively;

2) constructing cDNA libraries of the sample before stress treatment and after stress treatment respectively by using the total RNA of the sample obtained in step 1);

3) sequencing the cDNA libraries of the sample before stress treatment and after stress treatment in step 2) to obtain raw sequencing data of the sample before stress treatment and after stress treatment, respectively;

4) screening siRNAs from the raw sequencing data of the sample before stress treatment and after stress treatment to obtain siRNA data, respectively; combining the siRNA data of the sample before stress treatment and after stress treatment to obtain total siRNA data, and performing cluster clustering on the total siRNA data to obtain a total siRNA cluster annotation result, where the total siRNA cluster annotation result comprises positional information of the siRNA cluster and expression quantity information of the siRNA cluster;

5) repeat data in whole genome data is extracted by using repeatmasker software to obtain positional information of the plant whole genome transposon; and

6) screening siRNA clusters whose expression quantity changes before and after stress treatment from the total siRNA cluster in step 4), and aligning the positional information of the plant whole genome transposon in step 5) to positional information of the siRNA clusters whose expression quantity changes; if the expression quantity of the siRNA cluster at the position of the siRNA cluster corresponding to the position of a certain transposon changes, indicating that the transposon is activated; and if the expression quantity of the siRNA cluster at the position of the siRNA cluster corresponding to the position of a certain transposon does not change, indicating that the transposon is not activated.

Preferably, the plant is a Populus trichocarpa.

Preferably, the stress treatment comprises high-temperature stress treatment.

Preferably, the temperature of the high-temperature stress treatment is 38-42° C., and the time for the high temperature stress treatment is 8-16 h.

Preferably, the screening siRNAs from raw sequencing data in step 4) comprises the following steps:

4.1) screening 21-24 nt of small RNAs from the raw sequencing data; and

4.2) removing microRNA, tRNA, and rRNA from the screened small RNAs obtained in step 4.1) by using PatMaN software; using a mapper.pl program to align the small RNAs with the microRNA, tRNA, and rRNA removed to a reference genome; and screening the aligned small RNAs as siRNAs.

Preferably, the number of alignments in step 4.2) is 1,000, the number of misalignments is 0, and parameter selections of the mapper.pl program are as follows: mapper.pl -input -h -e -j -1 18 -m -r 1000 - p genome -n -v -o 20.

Preferably, the spacing of the cluster clustering in step 4) is 100-150 bp, and a tool for the cluster clustering is a Bedtools program.

Preferably, a tool for aligning the positional information of the plant whole genome transposon in step 6) to the positional information of the siRNA cluster whose expression quantity changes is a Bedtools program: bedtools intersect instruction.

Preferably, the expression quantity of the siRNA cluster in step 4) is the expression quantity of the siRNA having an internal expression quantity rpm greater than or equal to 5 in the siRNA cluster.

The advantageous effects of the present invention: the method for detecting activity change of a transposon in a plant before and after stress treatment provided by the present invention fills the technical gap in the field of plant transposon activity detections, and can accurately identify the activity changes in transposons before and after stress treatment. The method of the present invention accurately detects the amount of siRNA expressions and overcomes the quantitative inaccuracy caused by the large number of siRNAs, wide distribution, and large enrichment ratio in the conventional method.

### BRIEF DESCRIPTION OF THE DRAWINGS

FIG. 1 is a schematic flowchart of a method for detecting the activity of a transposon in a plant according to the present invention;

FIG. 2 is a diagram showing a classification ratio of transposons of Populus trichocarpa in Example 1 of the present invention before and after high-temperature stress treatment; and

FIG. 3 is a schematic diagram showing the plant morphology of Populus trichocarpa in Example 1 of the present invention before and after high-temperature stress treatment.

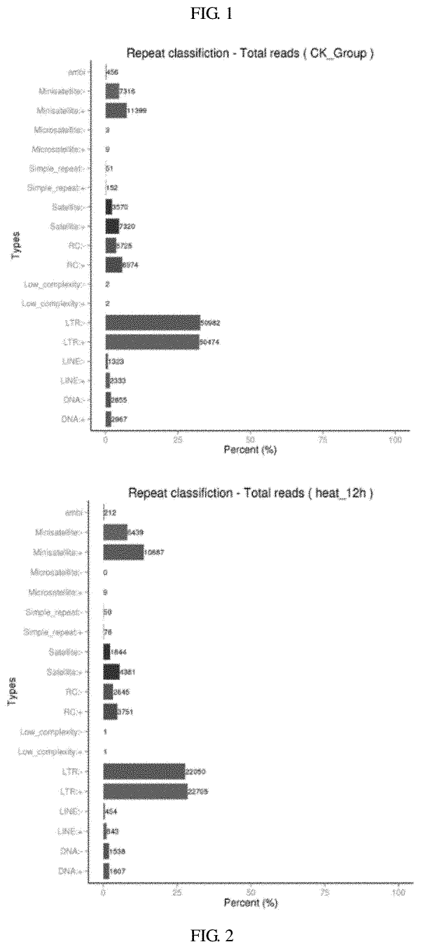

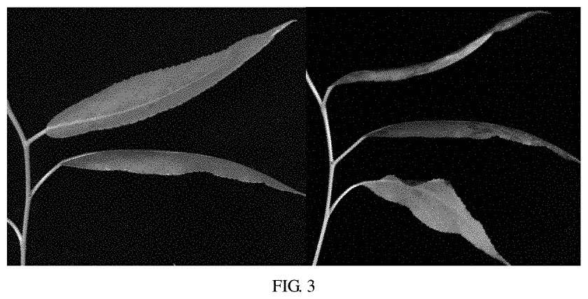

### DETAILED DESCRIPTION

The present invention provides a method for detecting activity change of a transposon in a plant before and after stress treatment, comprising the following steps:

1) total RNAs of a sample before stress treatment and after stress treatment are extracted respectively;

2) DNA libraries of the sample before and after stress treatment are respectively constructed by using the total RNA of the sample before stress treatment and after stress treatment obtained in step 1);

3) the cDNA libraries of the sample before stress treatment and after stress treatment in step 2) are respectively sequenced to obtain raw sequencing data of the sample before stress treatment and after stress treatment;

4) siRNAs are respectively screened from the raw sequencing data of the sample before stress treatment and after stress treatment to obtain siRNA data of the sample before stress treatment and after stress treatment; the siRNA data of the sample before stress treatment and after stress treatment are combined to obtain total siRNA data, and cluster clustering is performed on the total siRNA data to obtain a total siRNA cluster annotation result, where the total siRNA cluster annotation result comprises positional information of the siRNA cluster and expression quantity information of the siRNA cluster;

5) repeat data in whole genome data is extracted by using repeatmasker software to obtain positional information of the plant whole genome transposon; and

6) siRNA clusters whose expression quantity changes are screened from the total siRNA cluster in step 4), and the positional information of the plant whole genome transposon in step 5) is aligned to positional information of the siRNA clusters whose expression quantity changes; if the expression quantity of the siRNA cluster at the position of the siRNA cluster corresponding to the position of a certain transposon changes, it is indicated that the transposon is activated; and if the expression quantity of the siRNA cluster at the position of the siRNA cluster corresponding to the position of a certain transposon does not change, it is indicated that the transposon is not activated.

In the present invention, the method for detecting activity change of a transposon in a plant before and after stress treatment has no particular requirement on the species of the plant, and poplar is preferred. The poplar is preferably a model species, Populus trichocarpa. The stress treatment in the present invention is preferably a high-temperature stress treatment. The temperature of the high-temperature stress treatment is preferably 38-42° C., and more preferably 40° C. The time of the high-temperature stress treatment is preferably 8-16 h, more preferably 10-14 h, and most preferably 12 h. In the specific implementation of the present invention, the sample before stress treatment and after stress treatment are preferably leaf tissues of the sample before and after stress treatment of the same plant. In the present invention, the method of extracting a total RNA of the sample before stress treatment and after stress treatment is preferably a CTAB method. After obtaining the total RNA, the present invention detects the total quantity, purity and integrity of the total RNA. The purity determination method is specifically: RNase-free water is as a blank control, and A230, A260 and A280 values of the total RNA of each sample are respectively determined by using a spectrophotometer; the purity of the RNA sample is determined, and the total quantity thereof is calculated; the sample of qualified purity is selected for subsequent operations; and if the purity is not qualified, re-extraction is required. A260/A280 and A260/A230 are indicator values of the RNA purity. The ratio of A260/A280 at the pH of 7-8.5 is 1.8-2.0, indicating that the purity of RNA is good. The ratio of pure sample A260/A230 should be greater than 2.0 (RNA). If the ratio is less than 2.0, it is indicated the presence of protein or phenolic substances, and the total RNA of the sample needs to be re-extracted. The total quantity of RNAs is calculated by a conventional method in the art through measuring an OD value. In the present invention, the integrity detection is preferably performed by agarose gel electrophoresis. If three bands, i.e., 5S, 18S, and 35S appear, it is indicated that the RNA is an intact RNA.

In the present invention, after a total RNA of the sample before stress treatment and after stress treatment are obtained, cDNA libraries of the sample before stress treatment and after stress treatment are respectively constructed by using the total RNA of the sample before stress treatment and after stress treatment. In the present invention, the construction of the cDNA libraries of the sample before stress treatment and after stress treatment are preferably entrusted to a biological sequencing company. In the specific implementation of the present invention, Novogene Biological Information Technology Co., Ltd. is entrusted.

In the present invention, the cDNA libraries of the sample before stress treatment and after stress treatment are respectively sequenced. The sequencing is preferably entrusted to a biological sequencing company. In the specific implementation of the present invention, Novogene Biological Information Technology Co., Ltd. is entrusted. The read length of the sequencing in the present invention is preferably 50 nt. The sequencing is preferably 30× sequencing. The data volume of the sequencing is 10 M.

In the present invention, raw sequencing data sets of the sample before stress treatment and after stress treatment are obtained, siRNAs are respectively screened from the after the raw sequencing data of the sample before stress treatment and after stress treatment.

In the present invention, the screening siRNAs from the raw sequencing data preferably includes the following steps: screening 21-24 nt of small RNAs from the raw sequencing data; and removing microRNA, tRNA, and rRNA from the screened small RNAs by using PatMaN software; using a mapper.pl program to align the small RNAs with the microRNA, tRNA, and rRNA removed to a reference genome; and screening the aligned small RNAs as siRNAs. In the present invention, the screening criteria for screening siRNAs from the raw sequencing data are preferable as follows: the length of an siRNA mature sequence is generally between 21 nt and 24 nt; the siRNA mature sequence does not contain a stem-loop structure; an siRNA precursor is derived from double-stranded RNAs, transposons, and repeats; the free energy (MFE) of the siRNA mature sequence is less than −20 kcal/mol; and the siRNA mature sequence does not belong to snoRNA, rRNA, miRNA and tRNA. In the present invention, during genome alignment with the mapper.pl program, the number of alignments is preferably 1,000, the number of misalignments is 0, and parameter selections of the alignment are as follows: mapper.pl -input -h -e -j -1 18 -m -r 1000 -p genome -n -v -o 20.

In the present invention, after siRNAs of the samples before and after stress treatment are obtained, siRNA data of the sample before stress treatment and siRNA data of the sample after stress treatment are obtained; the siRNA data of the sample before stress treatment and the siRNA data of the sample after stress treatment are combined to obtain total siRNA data, and cluster clustering is performed on the total siRNA data to obtain a total siRNA cluster annotation result, where the total siRNA cluster annotation result comprises positional information of the siRNA cluster and expression quantity information of the siRNA cluster. In the present invention, the spacing of performing siRNA cluster clustering on the total siRNA is preferably 100-150 bp, and a tool for the cluster clustering is preferably a Bedtools program. In the specific implementation of the present invention, the used method and selection parameters are: bedtools merge -i input -c -o collapse, count, sum-d 100>output.

The present invention uses the repeatmasker software to extract the repeat in the whole genome data to obtain the positional information of the plant whole genome transposon. In the present invention, the repeatmasker software extracts the parameters of the repeat in the whole genome data by using RepeatMasker -no_is-pa 30 -species Populus -s -nolow -norna -dir repeat_pop -gff pop.fa.

In the present invention, siRNA clusters whose expression quantity changes before and after stress treatment are screened from the total siRNA cluster, and the positional information of the plant whole genome transposon is aligned to the positional information of the siRNA clusters whose expression quantity changes. If the expression quantity of the siRNA cluster at the position of the siRNA cluster corresponding to the position of certain transposon changes, it is indicated that the transposon is activated. If the expression quantity of the siRNA cluster at the position of the siRNA cluster corresponding to the position of a certain transposon does not change, it is indicated that the transposon is not activated.

In the present invention, the specific steps of screening the siRNA clusters whose expression quantity changes before and after the stress treatment are as follows:

an index file of the selected plant genome is constructed using a bowtie program; The second-generation sequencing transcriptome files are analyzed by a hisat2 process to obtain sam files before and after treatment.

The sam files are sorted. The first column is the chromosome, and the second column is the position start information.

In a Linux system, the sam files after sorting are processed by Stringtie software, and the total siRNA cluster annotation file obtained by the annotation files is selected to obtain the change in the expression quantity of the siRNA cluster of the plant before and after treatment, respectively. The selected parameters are stringtie input.sorted -e -G total_siRNA_cluster.gtf -p 7 -o output.

The foregoing files are screened, and the siRNA clusters with the expression quantity (rpm) greater than or equal to 5 are selected as the cluster clustering expression quantity of the sample.

In the specific implementation of the present invention, the step of aligning the positional information of the plant whole genome transposon to the positional information of the siRNA cluster whose expression quantity changes is preferably carried out by bedtools intersect of a Bedtools program.

The technical solution provided by the present invention will be described below in detail with reference to examples. However, the examples should not be construed as limiting the protection scope of the present invention.

### Example 1

The acquisition of raw materials: the annual individual of Populus trichocarpa is from the Li Wei research group of the Northeast Forestry University.

The various reagents used in the CTAB method are commercially available products.

siRNA evaluation is carried out using a Python code and a PatMaN software system.

Specific operation steps are as follows:

The annual individual of Populus trichocarpa is selected for stress treatment. FIG. 3 is an image showing the change in Populus trichocarpa before and after high-temperature treatment. The left side is the untreated Populus trichocarpa, and the right side is the Populus trichocarpa treated at 40 degrees for 12 h. To ensure that the RNA is not degraded, it is stored in a liquid nitrogen atmosphere (−196° C.) immediately after sampling.

The total RNA of a small number of samples is extracted by the CTAB method. The specific method is as follows:

0.1 g of plant tissue is added with an equal amount of PVPP (polypropylene pyrrolidone), ground in liquid nitrogen, and collected in a 50 ml centrifugal tube;

15 ml of (W:V=1:5) 65° C. pre-warmed CTAB extract (2% of CTAB, 4% of PVP, 25 mM of EDTA, 2.0 mM of NaCl, and 100 mM of Tris-HCl with the pH of 8.0) is added, and 300 μL of β-mercaptoethanol is added, vortexed and uniformly mixed, and subjected to water bath at 65° C. for 10 min.

An equal volume of chloroform:isoamylol (V:V=24:1) is added, and the mixture is gently extracted for 10 min and centrifuged at 12,000 rpm at 4° C. for 10 min. A supernatant is taken, and a 1/5 volume of 12 M LiCl is added and precipitated at 4° C. for 2 h.

The mixture is centrifuged at 12,000 rpm at 4° C. for 20 min. The supernatant is discarded, and 800 μL of LSSTE buffer solution is added for dissolving the precipitates. The buffer solution with the RNA dissolved is transferred to a 2 ml centrifugal tube.

An equal volume of chloroform:isoamylol is added, and the mixture is gently extracted for 5 min, and centrifuged at 12,000 rpm at 4° C. for 10 min. The supernatant is taken, and the mixture is repeatedly extracted twice.

The supernatant is taken, and 1/10 volume of 3 M NaAC (the pH of 5.2) and 2.5-fold volume of absolute ethanol are added, uniformly mixed, and then stand at −20° C. for 2 h to precipitate RNA. The mixture is centrifuged at 12,000 rpm at 4° C. for 20 min, the supernatant is discarded, and the precipitates are collected.

The DNA is removed with DNA digestive enzyme (1 μg of water-soluble RNA, 10 pt of 10×DNase reaction buffer, 10 μL of DNase, and RNase-free water to 50 μL) in a water bath at 37° C. for 30 min. An equal volume of 24:1 (chloroform:isoamylol) is added, mixed upside down, and then centrifuged at 12,000 rpm for 10 min.

The supernatant is dispensed into a 1.5 ml centrifugal tube, and then the 3-fold volume of absolute ethanol and 1/3 volume of 10 mol/L NaAC are added, and the mixture is uniformly mixed and stand at −20° C. for 2 h to precipitate the RNA. The mixture is centrifuged at 12,000 rpm at 4° C. for 20 min, the supernatant is discarded, and the precipitates are collected to obtain a total RNA extract.

Compared with the conventional method, the method for extracting total RNA in this embodiment reduces the step of extracting an equal volume of phenol:chloroform:isoamylol in the extraction step, which not only simplifies the test procedure but also achieves a good extraction effect. In addition, the concentration of LiCl added is 12 M, and the addition amount is 1/5 volume of the total volume of the supernatant, which changes the concentration and usage amount of LiCl compared with the conventional method. Through the improvement of the foregoing steps, the CTAB method provided by the present invention has the advantages of low required tissue amount, is suitable for the sampling of a small amount of tissue, and is advantageous for improving the accuracy of transcription analysis.

Finally, the purity, total amount and integrity of the extracted RNA are detected. Specifically, RNAse-free water is used as a blank control, and the A230, A260 and A280 values of each RNA sample are determined by a spectrophotometer to determine the purity of the RNA sample and calculate the total amount thereof. The integrity of the RNA sample is determined by gel electrophoresis, which meets the requirements of the sequencing company.

The cDNA library construction and sequencing steps are entrusted to Novogene Biological Technology Co., Ltd. for sequencing.

The specific steps are as follows: S1051, for the sequencing file of the tissue, the small RNAs of 21-24 nt size are screened; S1052, all the small RNAs of S1051 are annotated, the microRNA, tRNA, rRNA are removed, and the method used is preferably using the screened PatMaN, the calculation rate is fast, and Rfam, miBase, RepeatBase and other databases can be simultaneously aligned; S1053, genome alignment is performed on the file obtained in S1052 by using the mapper.pl program, the number of alignments is 1,000 times, the number of misalignments is 0, and parameter selections of the alignment are as follows: mapper.pl -input -h -e -j -1 18 -m -r 1000 -p genome -n -v -o 20; and S1054, siRNA cluster clustering is performed on the file obtained in S1053, the spacing is 100 bp to form a partition, i.e., a cluster, and the used method and selection parameters are: bedtools merge -i input -c -o collapse, count, sum-d 100>output.

According to the foregoing specific determining steps, the quantity distribution of the sample siRNAs before and after stress treatment and the cluster clustering result can be determined. Based on the siRNA distribution, quantity, and cluster clustering results, the total siRNA cluster annotation file is screened.

The change in expression quantity of siRNA clusters in different samples before and after stress treatment is obtained according to the total siRNA cluster annotation file. The specific implementation is as follows: S1061, the bowtie program is used to construct the index file of the selected plant genome; S1062, the second-generation sequencing transcriptome file is analyzed by the hisat2 process to obtain sam files before and after treatment, respectively; S1063, the sam files are sorted, the first column is the chromosome, the second column is the position start information; S1064, in the Linux system, the sorted sam files are treated by using the Stringtie software, the annotation files are selected as the total siRNA cluster annotation file obtained in S106 to obtain the change in expression quantity of siRNA clusters of the plant before and after treatment. The selected parameters are stringtie input.sorted -e -G total_siRNA_cluster.gtf -p 7 -o output; and S1065, the foregoing files are screened, and the siRNA clusters with the expression quantity (rpm) greater than or equal to 5 are screened as the cluster clustering expression quantity of the sample. Compared with the similar methods, the method used in each step of this step has the fastest calculation speed and the highest comparison rate, and the set parameters are all mismatched. Therefore, this step is particularly accurate in calculating the siRNA expression quantity.

According to the change in siRNA cluster expression quantity in the sample, the transposon enriched in the sample is obtained, and the activity change in transposon is deduced. As many studies show that the activation of the transposon results in the generation of siRNAs with the sizes of 21 nt, 22 nt, and 24 nt. The siRNA clustering expression quantity can clearly indicate the activity change in the transposon enriched in the region because the changes in the expression quantity of siRNA cluster before and after treatment are counted to obtain the activity change in transposon. The specific operation is as follows: S1071, the data files obtained in S106 are screened and aligned to obtain the siRNA cluster positional information expressed in the sample before and after the stress treatment, respectively; S1072, the repeat information is obtained using the repeatmasker software, and then the positional information of the plant whole genome transposon is obtained by screening; S1073, the method is used to combine the data files obtained in S1072 to obtain the expression quantity and positional information of the transposon in different samples; S1074, the positional information of S1071 and S1083 is enriched using a bedtools intersect of the Bedtools program; and S1075, the activity change level of transposon before and after Populus trichocarpa stress treatment is obtained through alignment and screening.

The specific results are shown in Tables 1-4:

TABLE 1
Classified statistical table of aligned siRNAs

| | | | Heat_12 h | |
|---|---|---|---|---|
| | | CK_Group | (the number | Heat_12 |
| Types | CK_Group | (percent) | of reads) | h(percent) |
| total | 10183731 | 100.00% | 10709856 | 100.00% |
| known_miRNA | 71897 | 0.71% | 25606 | 0.24% |
| rRNA | 1694459 | 16.64% | 1661295 | 15.51% |
| tRNA | 1 | 0.00% | 3 | 0.00% |
| snRNA | 7995 | 0.08% | 4136 | 0.04% |
| snoRNA | 19198 | 0.19% | 11379 | 0.11% |
| repeat | 155913 | 1.53% | 79493 | 0.74% |
| NAT | 783327 | 7.69% | 894440 | 8.35% |
| novel_miRNA | 2792 | 0.03% | 856 | 0.01% |
| TAS | 5 | 0.00% | 1 | 0.00% |
| exon: + | 1046103 | 10.27% | 1044525 | 9.75% |
| exon: − | 266483 | 2.62% | 373312 | 3.49% |
| intron: + | 329435 | 3.23% | 168559 | 1.57% |
| intron: − | 46304 | 0.45% | 42594 | 0.40% |
| other | 5759819 | 56.56% | 6403657 | 59.79% |

The quantity and classification of the small RNAs are accurately identified after the implementation of step S104, and the siRNAs are accurately screened.

TABLE 2
Clustering positional information of siRNA cluster (partial results)

| | | | | | The number of |
|---|---|---|---|---|---|
| | | Start | End | | enriched |
| | Chromosomes | position | position | Length | siRNAs |
| siRNA_cluster1 | Chr05 | 10693124 | 10694931 | 1807 | 25482 |
| siRNA_cluster2 | Chr05 | 10714250 | 10716057 | 1807 | 25482 |
| siRNA_cluster3 | Chr08 | 19184925 | 19186732 | 1807 | 25482 |
| siRNA_cluster4 | scaffold_346 | 47276 | 49083 | 1807 | 25482 |
| siRNA_cluster5 | Chr05 | 10783593 | 10785395 | 1802 | 24246 |
| siRNA_cluster6 | Chr05 | 10743145 | 10744941 | 1796 | 23905 |
| siRNA_cluster7 | Chr05 | 10734005 | 10735797 | 1792 | 21038 |
| siRNA_cluster8 | Chr05 | 10700548 | 10702325 | 1777 | 25283 |
| siRNA_cluster9 | Chr08 | 19156443 | 19158137 | 1694 | 24076 |
| siRNA_cluster10 | Chr13 | 14793584 | 14795075 | 1491 | 19702 |
| siRNA_cluster11 | Chr11 | 7214889 | 7216370 | 1481 | 14948 |
| siRNA_cluster12 | Chr05 | 10723523 | 10724986 | 1463 | 20644 |
| siRNA_cluster13 | Chr05 | 10792470 | 10793755 | 1285 | 17286 |
| siRNA_cluster14 | Chr14 | 16277320 | 16278590 | 1270 | 18128 |
| siRNA_cluster15 | scaffold_45 | 60183 | 61445 | 1262 | 12482 |
| siRNA_cluster16 | Chr08 | 19140802 | 19142062 | 1260 | 17286 |
| siRNA_cluster17 | Chr08 | 19159492 | 19160752 | 1260 | 17837 |
| siRNA_cluster18 | Chr14 | 16382920 | 16384179 | 1259 | 16859 |
| siRNA_cluster19 | Chr14 | 16266540 | 16267797 | 1257 | 16186 |
| siRNA_cluster20 | scaffold_45 | 37268 | 38525 | 1257 | 11234 |
| siRNA_cluster21 | scaffold_22 | 945332 | 946584 | 1252 | 13748 |
| siRNA_cluster22 | scaffold_346 | 78498 | 79750 | 1252 | 17769 |
| siRNA_cluster23 | Chr14 | 17059968 | 17061213 | 1245 | 8178 |
| siRNA_cluster24 | scaffold_45 | 229238 | 230483 | 1245 | 12164 |
| siRNA_cluster25 | Chr14 | 16445632 | 16446875 | 1243 | 15815 |
| siRNA_cluster26 | Chr14 | 16786531 | 16787774 | 1243 | 5244 |
| siRNA_cluster27 | Chr15 | 9394849 | 9396073 | 1224 | 5265 |
| siRNA_cluster28 | Chr10 | 5310246 | 5311446 | 1200 | 13169 |
| siRNA_cluster29 | Chr05 | 10764467 | 10765639 | 1172 | 16416 |
| siRNA_cluster30 | scaffold_22 | 1000103 | 1001144 | 1041 | 13978 |
| siRNA_cluster31 | Chr14 | 16418657 | 16419599 | 942 | 8686 |

Table 2 shows the partial results obtained after the implementation of step S104. Due to the huge amount of data, it is programmed by a Python code. Since the activity of the transposon needs to be identified, it is necessary to accurately quantify the expression quantity of siRNAs. However, since the quantity distribution of siRNAs is high in the genome, the length is short, and the coverage is large, it is extremely difficult to quantify a single siRNA and the error is easy to occur. The present invention recreates a method for the expression quantity of siRNAs, i.e., siRNA clustering, which is a partition per 100 bp, and is used to count the expression quantity of siRNAs expressed on the whole genome by using the expression quantity of the partition, thereby facilitating the definition of the activity of the transposon.

TABLE 3
Comparison of cluster expression quantity between the
two groups before and after treatment (partial results)

| | | | | CK | heat12 h |
|---|---|---|---|---|---|
| | | | | expression | expression |
| | | Start | End | quantity | quantity |
| | Chrorosomes | position | position | value | value |
| siRNAcluster4 | Chr01 | 125921 | 126136 | 1092.915 | 200.2084 |
| siRNAcluster10 | Chr01 | 574313 | 574333 | 35.83462 | 0 |
| siRNAcluster12 | Chr01 | 678227 | 678249 | 34.48713 | 0 |
| siRNAcluster37 | Chr01 | 3778877 | 3778971 | 143.155 | 109.9459 |
| siRNAcluster47 | Chr01 | 4848457 | 4848537 | 176.8535 | 53.51632 |
| siRNAcluster57 | Chr01 | 5710141 | 5710359 | 12851.99 | 10642.05 |
| siRNAcluster59 | Chr01 | 6041564 | 6041585 | 8.320324 | 0 |
| siRNAcluster70 | Chr01 | 7151405 | 7151427 | 20.33856 | 0 |
| siRNAcluster91 | Chr01 | 10736108 | 10736198 | 350.4156 | 157.2536 |
| siRNAcluster92 | Chr01 | 10736346 | 10736433 | 243.9472 | 127.4879 |
| siRNAcluster94 | Chr01 | 10919845 | 10919867 | 11.4957 | 11.66722 |
| siRNAcluster103 | Chr01 | 11862888 | 11863114 | 1760.047 | 1065.746 |
| siRNAcluster104 | Chr01 | 11863282 | 11863315 | 96.60819 | 6.67829 |
| siRNAcluster114 | Chr01 | 13128745 | 13128765 | 17.43306 | 0 |
| siRNAcluster124 | Chr01 | 14282312 | 14282343 | 1399.971 | 2477.149 |
| siRNAcluster176 | Chr01 | 18753162 | 18753182 | 273.5052 | 216.2005 |
| siRNAcluster181 | Chr01 | 19070966 | 19071027 | 216.3433 | 137.1695 |
| siRNAcluster188 | Chr01 | 19836126 | 19836150 | 89.8151 | 66.82507 |
| siRNAcluster199 | Chr01 | 21554401 | 21554436 | 15.44224 | 7.262934 |
| siRNAcluster215 | Chr01 | 23803989 | 23804141 | 147.6872 | 97.20634 |
| siRNAcluster219 | Chr01 | 24302887 | 24302910 | 14.40648 | 0 |
| siRNAcluster251 | Chr01 | 28316213 | 28316234 | 0 | 20.64201 |
| siRNAcluster252 | Chr01 | 28462309 | 28462375 | 1626.63 | 1073.231 |
| siRNAcluster267 | Chr01 | 30074570 | 30074664 | 115.9441 | 61.89706 |
| siRNAcluster268 | Chr01 | 30075919 | 30076065 | 4.261417 | 0.56169 |
| siRNAcluster277 | Chr01 | 31288963 | 31289020 | 6.873032 | 2.224363 |
| siRNAcluster279 | Chr01 | 31964689 | 31964716 | 6129.061 | 15012.92 |
| siRNAcluster309 | Chr01 | 35800378 | 35800400 | 1630.58 | 355.222 |

Table 3 shows the partial statistical results of cluster differential expression in the two samples obtained after the implementation of step S106, where the value of the expression quantity is a normalized value, and it can be seen that the siRNA expression quantity after 12 h of treatment at a high temperature of 40 degrees significantly changes, such as siRNAcluster279, siRNAcluster309, siRNAcluster92, and the like.

TABLE 4
Transposon activity comparison (partial results)

| | CK expression | heat12 h expression |
|---|---|---|
| repeat | quantity value | quantity value |
| repeat1736 | 930.0271 | 1292.002 |
| repeat4043 | 69.73222 | 106.1589 |
| repeat376 | 140.8504 | 46.3852 |
| repeat3736 | 0 | 38.10833 |
| repeat339 | 0 | 17.69316 |
| repeat768 | 36.76586 | 0 |
| repeat4255 | 17.79624 | 0 |
| repeat377 | 7.20032 | 0 |
| repeat1579 | 16.27085 | 0 |
| repeat2170 | 0 | 18.34846 |
| repeat3556 | 18.07873 | 0 |
| repeat3816 | 15.25392 | 0 |
| repeat2768 | 14.31233 | 0 |
| repeat2768 | 14.31233 | 0 |
| repeat4137 | 14.31233 | 0 |
| repeat4048 | 14.31233 | 0 |
| repeat1126 | 5.866903 | 0 |
| repeat2785 | 13.55905 | 0 |
| repeat2741 | 12.7116 | 0 |
| repeat925 | 11.86416 | 0 |
| repeat2901 | 0 | 5.897712 |

The results in Table 4 are the partial results of the activity change in transposon obtained after the implementation of the S107 step, and the value of the expression quantity is the normalized value of the expression quantity. The activity change in transposon of Populus trichocarpa after 12 h of treatment at a high temperature of 40 degrees is identified after the transposon information position screening and the siRNA cluster position enrichment.

It can be seen from the above experimental data that the screening method provided by the present invention has the following advantages: 1) the method fills in the blank of the identification method of Populus trichocarpa and even plant transposon activity, and can accurately identify the activity change in the plant transposon; 2) the method makes full use of the second-generation high-throughput sequencing technology, which can accurately perform high-throughput screening of siRNAs; 3) the step of siRNA quantification in the method corrects inaccurate quantification caused by the large quantity, wide distribution, and large enrichment proportion of siRNAs in the conventional methods; 4) compared with the conventional methods, the method requires a small number of tissues and is suitable for micro-tissue sampling, which is beneficial to improve the accuracy of transcription analysis.

The foregoing descriptions are only preferred implementation manners of the present invention. It should be noted that for a person of ordinary skill in the art, several improvements and modifications may further be made without departing from the principle of the present invention. These improvements and modifications should also be deemed as falling within the protection scope of the present invention.

1. A method for detecting activity change of a transposon in a plant before and after stress treatment, comprising the following steps:
1) respectively extracting total RNAs of a sample before stress treatment and after stress treatment;
2) respectively constructing cDNA libraries of the sample before stress treatment and after stress treatment by using the total RNA of the sample before stress treatment and after stress treatment obtained in step 1);
3) respectively sequencing the cDNA libraries of the sample before stress treatment and after stress treatment in step 2) to obtain raw sequencing data sets of the sample before stress treatment and after stress treatment;
4) respectively screening siRNAs from the raw sequencing data of the sample before stress treatment and after stress treatment to obtain siRNA data sets of the sample before stress treatment and after stress treatment; combining the siRNA data sets of the sample before stress treatment and after stress treatment to obtain total siRNA data, and performing cluster clustering on the total siRNA data to obtain a total siRNA cluster annotation result, wherein the total siRNA cluster annotation result comprises positional information of the siRNA cluster and expression quantity information of the siRNA cluster;
5) repeat in whole genome data is extracted by using repeatmasker software to obtain positional information of the plant whole genome transposon;
6) screening siRNA clusters whose expression quantity changes before and after stress treatment from the total siRNA cluster in step 4), and aligning the positional information of the plant whole genome transposon in step 5) to positional information of the siRNA clusters whose expression quantity changes; if the expression quantity of the siRNA cluster at the position of the siRNA cluster corresponding to the position of a certain transposon changes, indicating that the transposon is activated; and if the expression quantity of the siRNA cluster at the position of the siRNA cluster corresponding to the position of a certain transposon does not change, indicating that the transposon is not activated.

2. The method according to claim 1, wherein the plant is a Populus trichocarpa.

3. The method according to claim 2, wherein the stress treatment comprises high-temperature stress treatment.

4. The method according to claim 3, wherein the temperature of the high-temperature stress treatment is 38-42° C., and the time for the high-temperature stress treatment is 8-16 h.

5. The method according to claim 1, wherein the screening siRNAs from raw sequencing data in step 4) comprises the following steps:
4.1) screening 21-24 nt of small RNAs from the raw sequencing data;
4.2) removing microRNA, tRNA, and rRNA from the screened small RNAs obtained in step 4.1) by using PatMaN software; using a mapper.pl program to align the small RNAs with the microRNA, tRNA, and rRNA removed to a reference genome; and screening the aligned small RNAs as siRNAs.

6. The method according to claim 5, wherein the number of alignments in step 4.2) is 1,000, the number of misalignments is 0, and parameter selections of the mapper.pl program are as follows: mapper.pl -input -h -e -j -1 18 -m -r 1000 - p genome -n -v -o 20.

7. The method according to claim 1, wherein the spacing of the cluster clustering in step 4) is 100-150 bp, and a tool for the cluster clustering is a Bedtools program.

8. The method according to claim 1, wherein a tool for aligning the positional information of the plant whole genome transposon in step 6) to the positional information of the siRNA cluster whose expression quantity changes is a Bedtools program: bedtools intersect instruction.

9. The method according to claim 1, wherein the expression quantity of the siRNA cluster in step 4) is the expression quantity of the siRNA having an internal expression quantity rpm greater than or equal to 5 in the siRNA cluster.

10. The method according to claim 7, wherein the expression quantity of the siRNA cluster in step 4) is the expression quantity of the siRNA having an internal expression quantity rpm greater than or equal to 5 in the siRNA cluster.
